(19) United States
(12) Patent Application Publication
(10) Pub. No.: US 2009/0068117 A1
(43) Pub. Date: Mar. 12, 2009

(54) STAY-ON SELENIUM FOAM

(75) Inventors: JEFFREY S. DAY, Newtown, PA (US); MATS SILVANDER, Uppsala (SE)

Correspondence Address: Moser IP Law Group, 1030 BROAD STREET, 2ND FLOOR, SHREWSBURY, NJ, 07702, US

(73) Assignee: QUINNOVA PHARMACEUTICALS, INC., Newtown, PA (US)

(21) Appl. No.: 12/204,162

(22) Filed: Sep. 4, 2008

Related U.S. Application Data
(60) Provisional application No. 60/969,727, filed on Sep. 4, 2007.

(51) Int. Cl. A61K 9/12; A61K 33/04

(52) U.S. Cl. 424/45; 424/43; 424/702

(57) ABSTRACT

Provided, among other things, is a delivery module for a non-greasy, non-irritating selenium sulfide composition comprising: (A) an aerosol delivery device; (B) within the aerosol delivery device, the selenium sulfide composition comprising an effective amount of selenium sulfide that is 1% or more by weight of the composition, and a frothing agent, wherein the selenium sulfide composition has a viscosity low enough to support aerosol delivery and the selenium sulfide composition is effective to form a foam upon propellant-driven aerosol delivery; and (C) within the aerosol delivery device, a propellant.

This application is a non-provisional of U.S. Application No. 60/969,727, filed 2007 Sep. 4.

The present invention relates to foam-forming composition of selenium sulfide, which can be used to treat seborrheic dermatitis (“seb-derm”), tinea versicolor and the like.

The Merck Manual's Online Medical Library describes seb-derm as follows:
Seborrheic dermatitis is chronic inflammation of unknown cause that causes scales on the scalp and face and occasionally on other areas.
Seborrheic dermatitis occurs most often in infants, usually within the first 3 months of life, and between the ages of 30 and 70. The disorder is more common in men, often runs in families, and is worse in cold weather. A form of seborrheic dermatitis also occurs in as many as 85% of people with AIDS.
Seborrheic dermatitis usually begins gradually, causing dry or greasy scaling of the scalp (dandruff), sometimes with itching but without hair loss. In more severe cases, yellowish to reddish scaly pimples appear along the hairline, behind the ears, in the ear canal, on the eyebrows, on the bridge of the nose, around the nose, on the chest, and on the upper back. In infants younger than 1 month of age, seborrheic dermatitis may produce a thick, yellow, crusted scalp rash (cradle cap) and sometimes yellow scaling behind the ears and red pimples on the face. Frequently, a stubborn diaper rash accompanies the scalp rash. Older children and adults may develop a thick, tenacious, scaly rash with large flakes of skin.

The Merck Manual's Online Medical Library describes tinea versicolor as follows:
Tinea versicolor (pityriasis versicolor) is a fungal infection of the topmost layer of the skin causing scaly, discolored patches.
The infection, caused by the yeast Malassezia furfur, is quite common, especially in young adults. It rarely causes pain or itching, but it prevents areas of the skin from tanning, producing patches that are lighter in color than surrounding skin. People with naturally dark skin may notice lighter patches; people with naturally fair skin may get dark or lighter patches. The color depends on how the yeast affect the melanocytes, the cells that make the pigment (cross-reference omitted). The patches are often on the chest or back and may scale slightly. Over time, small areas can join to form large patches.

Seb-derm is caused by a fungus that, in some form, is found on the skin of a great majority of adults. Factors thought to be implicated in triggering seb-derm include heat, high humidity and immunodeficiency.

Both seb-derm and tinea-versicolor are treated with shampoos containing selenium sulfide, such as Selsun Blue™ shampoo, Head & Shoulders™ shampoo, Glo-Sel™ shampoo, Excel Lotion™ shampoo and the like. The 2.5% shampoos are often recommended for brief application, followed by rinsing. Or, longer treatments are recommended with long periods between treatments (such as a week). For example, Merck Manual Online describes treatments for tinea versicolor as including: selenium shampoo 2.5% (in 10-min applications daily for 1 wk or 24-h applications weekly for 1 mo). For 1% shampoos, MedicineNet.com mentions overnight treatments that are washed in the morning, and repeated for a week.

There is believed to be no foam-forming formulation of selenium sulfide, and no foam that is appropriate as a leave-on formulation—i.e. one that is not intended to be rinsed off shortly after application. Moreover, formulations of higher concentration selenium sulfide can be irritating, a property expected to be accentuated surfactant action present in a foam. Thus, a non-irritating leave on formulation is unexpected. In addition, the selenium sulfide delivered by the current foam is unusually well dispersed such that no visible particles are present on skin after application.

### SUMMARY OF THE INVENTION

Provided, among other things, is a delivery module for a non-greasy, non-irritating selenium sulfide composition comprising: (A) an aerosol delivery device; (B) within the aerosol delivery device, the selenium sulfide composition comprising an effective amount of selenium sulfide that is 1% or more by weight of the composition, and a frothing agent, wherein the selenium sulfide composition has a viscosity low enough to support aerosol delivery and the selenium sulfide composition is effective to form a foam upon propellant-driven aerosol delivery; and (C) within the aerosol delivery device, a propellant.

Also provided, among other things, is a selenium sulfide composition comprising, by weight: selenium sulfide 1-4%; fatty acid(s) and/or analogous alkyl amine(s) 1-7%; hydrophilic polymer(s) 0.2-3%; titrant, as needed in amount effective to substantially neutralize the fatty acid(s) or alkyl amine(s); frothing agent 0.3-2%; and humectant 0.5-10%, and optionally further comprising emollient(s) 0.5-3%.

Further provided, among other things, is a method of treating a dermatological disease treatable with selenium sulfide comprising applying to affected skin a foamed, non-greasy, non-irritating selenium sulfide composition comprising: an effective amount of selenium sulfide that is 1% or more by weight of the composition, and a frothing agent, wherein the selenium sulfide composition has a viscosity low enough to support aerosol delivery and the selenium sulfide composition is effective to form a foam upon propellant-driven aerosol delivery. The disease treated can be, for example, seborrheic dermatitis or tinea versicolor.

### DETAILED DESCRIPTION OF THE INVENTION

The selenium sulfide can be present at a relatively high concentration. (Unless a different meaning is specified, % concentrations are wt. percentages.) Selenium sulfide can be a single form or a mixture of forms. For example, selenium sulfide can be SeS₂, Se₄S₄, Se₂S₆, or a mixture thereof. Topical dosages can be, for example, from 0.5% to 10% by weight. In certain embodiments, a range beginning at 1%, or 2%, or 3% by weight is used. In certain embodiments, a range ending at 5%, or 4%, or 3%, or 2% by weight is used.

The composition can contain lipophilic components that are believed to help distribute selenium sulfide on and into the skin. A major portion of such lipophilic components can be amphiphatic compounds in amounts effective to stabilize the lipophilic components in solution and/or emulsified. Example amphiphatic compounds are fatty acids, which can be substantially or essentially ionized, wherein the salt is soluble in the aqueous solution of the selenium sulfide composition. Further examples are alkyl amines with one alkyl per amine having a size distribution analogous to that of an appropriate fatty acid composition. Further examples are nonionic detergents.

The fatty acid can, for example, be of any composition found in a natural source, including hydrolysis of esterified fatty acids. Or, the fatty acid component can be hydrogenated to remove substantially all or a portion of any unsaturation. In certain embodiments, the fatty acid component is selected such that 50 mole % or more is C12 or higher, or C14, or C16 or higher. In certain embodiments, the fatty acid component is selected such that 50 mole % or more is C22 or lower, or C20 or lower, or C18 or lower. In certain embodiments, 75 mole % or more of the fatty acid component is from C12 or C14 or C16 to C22 or C20 or C18. In certain embodiments, 80 mole % or more, 85 mole % or more, 90 mole % or more, 95 mole % or more, 97 mole % or more, 98 mole % or more, or 99 mole % or more, meets one of the size parameters of this paragraph (e.g., C22 or lower).

For carboxylic acid containing lipophilic components, useful salts include the alkali metal salts such as sodium or potassium salts; ammonium salts; salts formed with suitable organic bases, such as amine salts (such as triethyl amine, triethanol amine, or the like) and quaternary ammonium salts; or the like. Bivalent or trivalent salts can be used where they do not adversely affect solubility. For amine-containing lipophilic components, useful salts include maleates, fumarates, lactates, oxalates, methanesulfonates, ethanesulfonates, benzenesulfonates, tartrates, citrates, halides (e.g., hydrochlorides, hydrobromides), sulfates, phosphates, nitrates, or the like. As needed, the lipophilic components are provided such that a sufficient amount of constituent ionizable molecules are in ionized (salt) form to provide solubility. Such ionized forms can be prepared by adding a titrant, though recitations of compositions described by such titration include the equivalent compositions formed by pre-formed salts or otherwise.

The lipophilic component may include 50% or less of a more hydrophobic component, such as one that can be termed an emollient. This more hydrophobic component can be, for example, 45% or less, or 40% or less, or 35% or less, or 30% or less, or 25% or less, or 20% or less, of the lipophilic component.

In certain embodiments, the lipophilic component is 1% or more, or 1.5% or more, or 2% or more, or 2.5% or more, or 3%, or 3.5% or more, or 4%, or 4.5% or more, or 5% or more of the selenium sulfide composition. In certain embodiments, the lipophilic component is 10% or less, 9.5% or less, 9% or less, 8.5% or less, 8% or less, 7.5% or less, 7% or less, 7.5% or less, 6% or less, 5.5% or less, 5% or less, or 4.5% or less, or 4% or less, or 3.5% or less of the selenium sulfide composition. Where the lipophilic component comprises, as predominant component(s), fatty acids or analogous alkyl amines, these predominate components can be 1% or more, or 1.5% or more, or 2% or more, or 2.5% or more, or 3%, or 3.5% or more, or 4% or more of the selenium sulfide composition; and 7% or less, or 6.5% or less, or 6% or less, or 5.5% or less, 5% or less, or 4.5% or less, or 4% or less, or 3.5% or less of the selenium sulfide composition.

An emollient, if present, can be a silicone oil such as polydimethylsiloxane (i.e., dimethicone), petrolatum, or the like. In certain embodiments, the emollient(s) are 0.5% or more, or 0.6% or more, or 0.7% or more, or 0.8% or more, or 0.9% or more, or 1% or more of the selenium sulfide composition. In certain embodiments, the emollient(s) are 3% or less, or 2.9% or less, or 2.8% or less, or 2.7% or less, or 2.6% or less, or 2.5% or less, or 2.4% or less, or 2.3% or less, or 2.2% or less, or 2.1% or less, 2% or less, or 1.9% or less, or 1.8% or less, or 1.7% or less, or 1.6% or less, or 1.5% or less, or 1.4% or less, or 1.3% or less, or 1.2% or less, or 1.1% or less, or 1% or less of the selenium sulfide composition.

In certain embodiments, the wt. ratio of the lipophilic component to frothing agent is from 2.7 to 3.7, such as from one of 2.7, 2.8, 2.9, 3.0, 3.1 or 3.2 to one of 3.1, 3.2, 3.3, 3.4, 3.5, 3.6 or 3.7. In certain embodiments, the wt. ratio of the fatty acid to frothing agent is from 2.0 to 3.0, such as from one of 2.0, 2.1, 2.2, 2.3, 2.4 or 2.5 to one of 2.4, 2.5, 2.6, 2.7, 2.8, 2.9 or 3.0.

A non-greasy feel is measured in reference to oil-based ointments and by comparison of the feel of the Example composition (described in the Example below), applied to skin at 1 mg/cm², compared to the oil-based product described in the Table at Column 3 of U.S. Pat. No. 5,919,470 (Bradley Pharmaceuticals, Inc.), applied in the same amount. While the feel of compositions of the invention may vary, in making the comparison between the non-greasy standard, the greasy standard, and the prospective non-greasy composition, it will be apparent which category the prospective composition falls within. The non-greasy skin feel may be moist and smooth feeling, but the difference in greasy feel relative to the greasy comparative shall be clear. In certain embodiments, the non-greasy comparative is the Example 1 composition (non-greasy standard) of U.S. application Ser. No. 12/016,371, filed Jan. 18, 2008 (US2008/0175793).

The composition can contain hydrophilic polymer(s). Hydrophilic polymer(s) can be any non-toxic water soluble polymer(s) that (in the aggregate) stabilize foam and contribute to film formation on the skin. Examples include polyvinyl pyrrolidone, polyethylene glycol, starch, water-soluble derivatives of starch, cellulose, methyl cellulose, hydroxymethylcellulose, other water-soluble derivatives of cellulose, carbomers, or the like. For polyvinyl pyrrolidone, for example, useful average molecular weights include from 8,000 to 63,000, such as about 38,000. For all polymers used in the composition, the size should be sufficient to limit penetration of the horny layer of the skin, if skin penetration is an issue for the given polymer.

In certain embodiments, hydrophilic polymer(s) are 0.2% or more, 0.3% or more, 0.4% or more, 0.5% or more, or 0.6% or more, or 0.7% or more, or 0.8% or more, or 0.9% or more, or 1% or more, or 1.5% or more of the selenium sulfide composition. In certain embodiments, the hydrophilic polymer(s) are 3% or less, 2.5% or less, 2% or less, 1.5% or less, or 1.4% or less, or 1.3% or less, or 1.2% or less, or 1.1% or less, or 1% or less of the selenium sulfide composition.

The composition can also contain a humectant, such as glycerol, propylene glycol, other polyols, polydextrose, lactic acid, or the like. In certain embodiments, humectant(s) are 0.5% or more, or 0.6% or more, or 0.7% or more, or 0.8% or more, or 0.9% or more, or 1% or more, or 1.2% or more, or 1.4% or more, or 1.6% or more, or 1.8% or more, or 2% or more, or 2.5% or more, or 3% or more, or 3.5% or more, or 4% or more, or 4.5% or more, or 5% or more, or 5.5% or more, or 6% or more of the selenium sulfide composition. In certain embodiments, the humectant(s) are 10% or less, or 9.5% or less, 8% or less, or 7.5% or less, 7% or less, or 6.5% or less, or 6.0% or less, or 5.8% or less, or 5.6% or less, or 5.4% or less, or 5.2% or less, or 5% or less of the selenium sulfide composition.

The frothing agent can be, for example, a non-ionic detergent such as Polysorbate 20, polyoxyethylene sorbitan fatty acid esters, sorbitol fatty acid esters, or the like. In certain embodiments, the frothing agent(s) are 0.3% or more, or 0.4% or more, or 0.5% or more, or 0.6% or more, or 0.7% or more, or 0.8% or more, or 0.9% or more, or 1% or more of the selenium sulfide composition. In certain embodiments, the frothing agent(s) are 2% or less, 1.5% or less, or 1.4% or less, or 1.3% or less, or 1.2% or less, or 1.1% or less, or 1% or less, or 0.9% or less, or 1.8% or less of the selenium sulfide composition.

In certain embodiments, the selenium sulfide composition can contain soothing agent(s) such as homogenized oatmeal. In certain embodiments, the soothing agent(s) are 0.02% or more, 0.03% or more, 0.04% or more, 0.05% or more, or 0.06% or more, or 0.07% or more, or 0.08% or more, or 0.09% or more, or 0.01% or more of the selenium sulfide composition. In certain embodiments, the soothing agent(s) are 0.2% or less, or 0.15% or less, or 0.14% or less, or 0.13% or less, or 0.12% or less, or 0.11% or less, or 1% or less of the selenium sulfide composition.

Additional optional ingredients include sunscreens, antimicrobial agents or preservatives, fragrances, and the like.

Suitable propellants include, for example, propane, butane, isobutene, other hydrocarbons, hydrofluorocarbons, chlorofluorocarbons (CI/F/(H)/C), and the like.

The amount of selenium sulfide composition applied to an affected area of skin can vary with a number of variables including the condition of the skin, the sensitivity of the patient or the area of skin, and the like. In any single administration, the delivery device can deliver to the affected area an appropriate layer of foam that provides an appropriate amount of selenium sulfide composition. The aerosol-driven foam can be applied to the affect area and rubbed into the skin until absorbed. Typically, the composition is applied twice a day. The foam is rubbed into the skin until completely absorbed.

In certain embodiments, the formulation of the invention provides a non-irritating foam. Irritation is measured by ISO 10993-10: 2002 Standard, “Biological Evaluation of Medical Devices, Part 10-Tests for Irritation and Sensitization,” pp. 6-10, 21, which testing method is incorporated herein by reference. In particular, for each test site on shaved dorsal skin of an albino rabbit, gauze incorporating 0.5 mL of test material or negative control material is applied. One test and one control site are used on each side of the paravertebral skin. The infused gauzes are covered with tape-backed gauze. The trunk of the rabbit is wrapped in elastic bandage secured by hypoallergenic tape. After a minimum of 24 hours, the coverings are unwrapped. Observations are made at 60 min±2, 24 h±2, 48 h±2 and 72 h±2 post unwrapping. Tissue reactions are rated for gross evidence of erythema and edema.

For a given rabbit, values for each test site and each of the 24 h, 48 h and 72 h measurements are totaled, and divided by six (2 tests sites X 3 measurements). Control values were treated in the same way. For all rabbits, these test values were summed, normalized against the summed values for the negative controls, and divided by the number of animals. A negligible, slight, moderate or severe response is categorized based on the Primary Irritation Index:

| Response Category | Comparative Mean Score |
|---|---|
| Negligible | 0 to 0.4 |
| Slight | 0.5 to 1.9 |
| Moderate | 2 to 4.9 |
| Severe | 5 to 8 |

By “non-irritating” it is meant that compositions according to this embodiment of the invention illicit a Negligible Primary Irritation Index.

In certain embodiments, the foam of the invention has a “non-watery feel” when applied. A non-watery feel is a feel much like that of the Example 1 composition (non-watery standard) of U.S. application Ser. No. 12/016,371, filed Jan. 18, 2008 (US2008/0175793), applied to skin at 1 mg/cm². A feel that, in contrast, is substantially more watery, is disqualified.

In certain embodiments, the foam-forming composition of the invention is essentially free of C1 to C6 alcohols (not including glycols or glycerin). By essentially free it is meant that such alcohols may be present in minor amounts, as may be useful for example for compounding, but are not present in an amount that one of skill in the art of pharmaceutical foam formulating would select to stabilize the selenium or the emulsion of a foam-forming composition. In certain embodiments, the amount of such alcohols is less than about 5 wt %, or 4 wt %, or 3 wt %, or 2 wt %, or 1 wt %, or 0.5 wt %.

The leave-on foam typically has a detergent content selected to, when mixed with the lipophilic component, not be strong enough for use as a shampoo.

### EXAMPLE

The following compositions can be formulated:

| Component | Wt. % (one of) |
|---|---|
| Povidone (PVP) | 1.0, 1.1, 1.2, 1.3, 1.4, 1.5, 1.6, 1.7, |
| | 1.8, 1.9, 2.0, 2.1, 2.2, 2.3, 2.4, 2.5 |
| Stearic acid | 4.5, 4.6, 4.7, 4.8, 4.9, 5.0, 5.1, |
| | 5.2, 5.3, 5.4, 5.5 |
| Glycerin | 1.0, 1.1, 1.2, 1.3, 1.4, 1.5, 1.6, |
| | 1.7, 1.8, 1.9, 2.0 |
| Dimethicone | 1.8, 1.85, 1.9, 1.95, 2.0, 2.05, 2.1, 2.15 |
| Propylene glycol | 5.0, 5.1, 5.2, 5.3, 5.4, 5.5, 5.6, |
| | 5.7, 5.8, 5.9, 6.0 |
| Triethanoleamine | To yield a pH from 6 to 9.5 |
| Polysorbate 20 | 1.0, 1.1, 1.2, 1.3, 1.4, 1.5, 1.6, |
| | 1.7, 1.8, 1.9, 2.0 |
| Mixture of methyl paraben, ethyl | 0.5 |
| paraben, propyl paraben and 2- | |
| ethoxyethanol (as preservative) | |
| Selenium sulfide | 2.25 |
| Water | To make 100% |
| Total | 100.00 |

In main kettle water, pvp, and stearic acid are heated to 70° C. In separate kettle propylene glycol, glycerin, dimethicone and selenium sulfide are mixed or homogenized. The selenium sulfide mix is then added to main kettler while stirring. After that trolamine, Phenonipe™ (mixture of methyl paraben, ethyl paraben, propyl paraben and 2-ethoxyethanol), and finally polysorbate 20 are added.

### DEFINITIONS

The following terms shall have, for the purposes of this application, the respective meanings set forth below.

Effective Amount

To treat the indications of the invention, an effective amount of a selenium sulfide will be recognized by clinicians but includes an amount effective to treat, reduce, alleviate, ameliorate, eliminate or prevent one or more symptoms of the disease sought to be treated or the condition sought to be avoided or treated, or to otherwise produce a clinically recognizable favorable change in the pathology of the disease or condition. In effective amount can be a dermatological treatment effective concentration of selenium sulfide.

### FURTHER ILLUSTRATIVE EMBODIMENTS

### Embodiment 1

A delivery module for a non-greasy, non-irritating selenium sulfide composition comprising:

an aerosol delivery device;

within the aerosol delivery device, the selenium sulfide composition comprising an effective amount of selenium sulfide that is 1% or more by weight of the composition, and a frothing agent, wherein the selenium sulfide composition has a viscosity low enough to support aerosol delivery and the selenium sulfide composition is effective to form a foam upon propellant-driven aerosol delivery; and

within the aerosol delivery device, a propellant.

### Embodiment 2

The delivery module of embodiment 1, wherein the selenium sulfide composition comprises, by weight:

selenium sulfide 1-4%;

fatty acid(s) and/or analogous alkyl amine(s) 1-7%;

hydrophilic polymer(s) 0.2-3%;

titrant, as needed in amount effective to substantially neutralize the fatty acid(s) or alkyl amine(s);

frothing agent(s) 0.3-2%; and

humectant(s) 0.5-10%.

### Embodiment 3

The delivery module of embodiment 1, wherein the selenium sulfide composition comprises, by weight:

selenium sulfide 1-4%;

fatty acid(s) and/or analogous alkyl amine(s) 1-7%;

emollient(s) 0.5-3%

hydrophilic polymer(s) 0.2-3%;

titrant, as needed in amount effective to substantially neutralize the fatty acid(s) or alkyl amine(s);

frothing agent(s) 0.3-2%; and

humectant(s) 0.5-10%.

### Embodiment 4

The delivery module of one of embodiments 1-3, wherein the selenium sulfide composition comprises, by weight, 4.5%-5.5% fatty acid(s).

### Embodiment 5

The delivery module of one of embodiments 1-4, wherein the selenium sulfide composition comprises, by weight, 1.0%-2.0% frothing agent(s).

### Embodiment 6

The delivery module of one of embodiments 1-5, wherein the selenium sulfide composition comprises, by weight, 1.0%-2.0% emollient(s).

### Embodiment 7

A selenium sulfide composition comprising, by weight:

selenium sulfide 1-4%;

fatty acid(s) and/or analogous alkyl amine(s) 1-7%;

hydrophilic polymer(s) 0.2-3%;

titrant, as needed in amount effective to substantially neutralize the fatty acid(s) or alkyl amine(s);

frothing agent(s) 0.3-2%; and

humectant 0.5-10%.

### Embodiment 8

The selenium sulfide composition of embodiment 7, comprising:

selenium sulfide 1-4%;

fatty acid(s) and/or analogous alkyl amine(s) 1-7%;

emollient(s) 0.5-3%;

hydrophilic polymer(s) 0.2-3%;

titrant, as needed in amount effective to substantially neutralize the fatty acid(s) or alkyl amine(s);

frothing agent(s) 0.3-2%; and humectant(s) 0.5-10%.

### Embodiment 9

The selenium sulfide composition of one of embodiments 7 to 8, wherein the selenium sulfide composition comprises, by weight, 4.5%-5.5% fatty acid(s).

### Embodiment 10

The selenium sulfide composition of one of embodiments 7 to 9, wherein the selenium sulfide composition comprises, by weight, 1.0%-2.0% frothing agent(s).

### Embodiment 11

The selenium sulfide composition of one of embodiments 7 to 10, wherein the selenium sulfide composition comprises, by weight, 1.0%-2.0% emollient(s).

### Embodiment 12

A method of treating dermatological disease treatable with selenium sulfide comprising applying to affected skin a foamed, non-greasy, non-irritating selenium sulfide composition comprising: an effective amount of selenium sulfide that is 1% or more by weight of the composition, and a frothing agent, wherein the selenium sulfide composition has a viscosity low enough to support aerosol delivery and the selenium sulfide composition is effective to form a foam upon propellant-driven aerosol delivery.

### Embodiment 13

The method of treating of embodiment 12, wherein the composition applied comprises:

selenium sulfide 1-4%;

fatty acid(s) and/or analogous alkyl amine(s) 1-7%;

hydrophilic polymer(s) 0.2-3%;

titrant, as needed in amount effective to substantially neutralize the fatty acid(s) or alkyl amine(s);

frothing agent(s) 0.3-2%; and

humectant(s) 0.5-10%.

### Embodiment 14

The method of one of embodiments 12 to 13, wherein the composition applied comprises:

selenium sulfide 1-4%;

fatty acid(s) and/or analogous alkyl amine(s) 1-7%;

emollient(s) 0.5-3%

hydrophilic polymer(s) 0.2-3%;

titrant, as needed in amount effective to substantially neutralize the fatty acid(s) or alkyl amine(s);

frothing agent(s) 0.3-2%; and

humectant(s) 0.5-10%.

### Embodiment 15

The method of treating of one of embodiments 12 to 14, wherein the disease treated is seborrheic dermatitis or tinea versicolor.

Publications and references, including but not limited to patents and patent applications, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

While this invention has been described with an emphasis upon preferred embodiments, it will be obvious to those of ordinary skill in the art that variations in the preferred devices and methods may be used and that it is intended that the invention may be practiced otherwise than as specifically described herein. Accordingly, this invention includes all modifications encompassed within the spirit and scope of the invention as defined by the claims that follow.

What is claimed:

1. A delivery module for a non-greasy, non-irritating selenium sulfide composition comprising:
an aerosol delivery device;
within the aerosol delivery device, the selenium sulfide composition comprising an effective amount of selenium sulfide that is 1% or more by weight of the composition, and a frothing agent, wherein the selenium sulfide composition has a viscosity low enough to support aerosol delivery and the selenium sulfide composition is effective to form a foam upon propellant-driven aerosol delivery; and
within the aerosol delivery device, a propellant.

2. The delivery module of claim 1, wherein the selenium sulfide composition comprises, by weight:
selenium sulfide 1-4%;
fatty acid(s) and/or analogous alkyl amine(s) 1-7%;
hydrophilic polymer(s) 0.2-3%;
titrant, as needed in amount effective to substantially neutralize the fatty acid(s) or alkyl amine(s);
frothing agent(s) 0.3-2%; and
humectant(s) 0.5-10%.

3. The delivery module of claim 1, wherein the selenium sulfide composition comprises, by weight, 4.5%-5.5% fatty acid(s).

4. The delivery module of claim 3, wherein the selenium sulfide composition comprises, by weight, 1.0%-2.0% frothing agent(s).

5. The delivery module of claim 1, wherein the selenium sulfide composition comprises, by weight:
selenium sulfide 1-4%;
fatty acid(s) and/or analogous alkyl amine(s) 1-7%;
emollient(s) 0.5-3%
hydrophilic polymer(s) 0.2-3%;
titrant, as needed in amount effective to substantially neutralize the fatty acid(s) or alkyl amine(s);
frothing agent(s) 0.3-2%; and
humectant(s) 0.5-10%.

6. The delivery module of claim 5, wherein the selenium sulfide composition comprises, by weight, 4.5%-5.5% fatty acid(s).

7. The delivery module of claim 6, wherein the selenium sulfide composition comprises, by weight, 1.0%-2.0% frothing agent(s).

8. The delivery module of claim 7, wherein the selenium sulfide composition comprises, by weight, 1.0%-2.0% emollient(s).

9. A selenium sulfide composition comprising, by weight:
selenium sulfide 1-4%;
fatty acid(s) and/or analogous alkyl amine(s) 1-7%;
hydrophilic polymer(s) 0.2-3%;
titrant, as needed in amount effective to substantially neutralize the fatty acid(s) or alkyl amine(s);
frothing agent(s) 0.3-2%; and
humectant 0.5-10%.

10. The selenium sulfide composition of claim 9, wherein the selenium sulfide composition comprises, by weight, 4.5%-5.5% fatty acid(s).

11. The selenium sulfide composition of claim 10, wherein the selenium sulfide composition comprises, by weight, 1.0%-2.0% frothing agent(s).

12. The selenium sulfide composition of claim 9, comprising:
selenium sulfide 1-4%;
fatty acid(s) and/or analogous alkyl amine(s) 1-7%;
emollient(s) 0.5-3%;
hydrophilic polymer(s) 0.2-3%;
titrant, as needed in amount effective to substantially neutralize the fatty acid(s) or alkyl amine(s);
frothing agent(s) 0.3-2%; and
humectant(s) 0.5-10%.

13. The selenium sulfide composition of claim 12, wherein the selenium sulfide composition comprises, by weight, 4.5%-5.5% fatty acid(s).

14. The selenium sulfide composition of claim 13, wherein the selenium sulfide composition comprises, by weight, 1.0%-2.0% frothing agent(s).

15. The selenium sulfide composition of claim 14, wherein the selenium sulfide composition comprises, by weight, 1.0%-2.0% emollient(s).

16. A method of treating dermatological disease treatable with selenium sulfide comprising applying to affected skin a foamed, non-greasy, non-irritating selenium sulfide composition comprising:
an effective amount of selenium sulfide that is 1% or more by weight of the composition, and a frothing agent, wherein the selenium sulfide composition has a viscosity low enough to support aerosol delivery and the selenium sulfide composition is effective to form a foam upon propellant-driven aerosol delivery.

17. The method of treating of claim 16, wherein the disease treated is seborrheic dermatitis or tinea versicolor.

18. The method of treating of claim 16, wherein the composition applied comprises:
selenium sulfide 1-4%;
fatty acid(s) and/or analogous alkyl amine(s) 1-7%;
hydrophilic polymer(s) 0.2-3%;
titrant, as needed in amount effective to substantially neutralize the fatty acid(s) or alkyl amine(s);
frothing agent(s) 0.3-2%; and
humectant(s) 0.5-10%.

19. The method of treating of claim 18, wherein the disease treated is seborrheic dermatitis or tinea versicolor.

20. The method of claim 16, wherein the composition applied comprises:
selenium sulfide 1-4%;
fatty acid(s) and/or analogous alkyl amine(s) 1-7%;
emollient(s) 0.5-3%
hydrophilic polymer(s) 0.2-3%;
titrant, as needed in amount effective to substantially neutralize the fatty acid(s) or alkyl amine(s);
frothing agent(s) 0.3-2%; and
humectant(s) 0.5-10%.
